(19) United States
(12) Patent Application Publication
(10) Pub. No.: US 2022/0282150 A1
(43) Pub. Date: Sep. 8, 2022

(54) LIGHT EMITTING MARKER AND ASSAY METHOD

(71) Applicant: Sumitomo Chemical Co., Ltd., Tokyo (JP)

(72) Inventors: Melanie O'Sullivan, Godmanchester (GB); Jonathan Behrendt, Godmanchester (GB)

(73) Assignee: Sumitomo Chemical Co., Ltd., Tokyo (JP)

(21) Appl. No.: 17/633,427

(22) PCT Filed: Aug. 6, 2020
(86) PCT No.: PCT/GB2020/051887

§ 371 Date: Feb. 7, 2022

(30) Foreign Application Priority Data
Aug. 8, 2019 (GB) 1911345.5

(51) Int. Cl. C09K 11/02; G01N 33/531; G01N 33/543; C09K 11/06

(52) U.S. Cl. CPC C09K 11/025; G01N 33/531; G01N 33/54346; C09K 11/06

(57) ABSTRACT

A light-emitting marker having a light-emitting core comprising a light-emitting material bound to a first biotin group and a biomolecule bound to a second biotin group. A protein, e.g. streptavidin or neutravidin, is bound to the first and second biotin groups. The light-emitting marker may be a light-emitting marker particle having a particulate core.

### BACKGROUND

In some embodiments, the present disclosure provides light-emitting markers, optionally light-emitting marker particles, for use as markers in biosensor applications.

Nanoparticles of silica and a light-emitting material have been disclosed as labelling or detection reagents.

WO 2018/060722 discloses composite particles comprising a mixture of silica and a light-emitting polymer having polar groups.

Estevez, M-C. et al. ‘Highly Fluorescent Dye-Doped Silica Nanoparticles Increase Flow Cytometry Sensitivity for Cancer Cell Monitoring’ Nano. Res. 2009, 2, 448-461 discloses dye-doped silica nanoparticles functionalised with polyethylene glycol.

US 2010/209946 discloses silica nanoparticles functionalised with water dispersible groups, shielding groups and biomolecule binding groups.

Pellegrino et al, “Multiple Dye Doped Core-Shell Silica Nanoparticles: Outstanding Stability and Signal Intensity Exploiting FRET Phenomenon for Biomedical Application” J. Nanomater. Mol. Nanotechnol. 2018, S6; doi: 10.4172/2324-8777.S6-003 discloses core-shell silica nanoparticles doped with different dyes entrapped in a silica core and conjugated with Anti-Human CD8 antibody.

US2013/0183665 discloses a process for the production of fluorescent nanoparticles selected from noble metal, silica or polymer nanoparticles.

### SUMMARY

A summary of aspects of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects and/or a combination of aspects that may not be set forth.

In some embodiments there is provided an assay method for a target analyte comprising contacting a sample with a light-emitting marker and determining any binding of the target analyte to the light-emitting marker wherein the light-emitting marker has a light-emitting core containing or consisting of a light-emitting material. A first group comprising a first biotin group is bound to the light-emitting core. A second biotin group is bound to a biomolecule. A protein is bound to the first and second biotin groups.

Optionally, the biomolecule comprises an antigen-binding fragment. The biomolecule may be an antibody.

Optionally, the protein is selected from avidin, streptavidin, neutravidin and recombinant variants thereof.

Optionally, the light-emitting core comprises or consists of a light-emitting polymer.

In some embodiments, the light-emitting marker is dispersed in the sample.

Optionally, the light-emitting marker is a light-emitting particle having a particulate light-emitting core containing the light-emitting material and wherein a first surface group bound to a surface of the light-emitting core includes the first biotin group.

Optionally, the first surface group has a polyether chain disposed between the surface of the light-emitting core and the first biotin group.

Optionally, the polyether group is a group of formula (I):

—((CR¹⁴R¹⁵)_{b}O)_{c}— (I)

wherein R¹⁴ and R¹⁵ are each independently H or C₁₋₆ alkyl; b is at least 1; and c is at least 2.

In some embodiments, the light-emitting marker is dissolved in the sample.

Optionally, a second surface group is bound to the surface of the light-emitting core wherein the second surface group does not comprise biotin.

Optionally, the second surface group comprises a polyether.

Optionally, the first surface group: second surface group molar ratio is in the range of 1:1000-1:10.

Optionally, the light-emitting core contains the light-emitting material and a matrix material.

Optionally, the matrix material is silica.

Optionally, the target analyte is a target antigen.

Optionally, the sample contacted with the light-emitting marker is analysed by flow cytometry.

Optionally, an amount of target analyte bound to the light-emitting marker is determined, e.g. determined by the flow cytometry analysis.

Optionally, the sample comprises a mixture of cells and one or more different types of target cells bound to the light-emitting marker are identified and/or quantified.

In some embodiments, the present disclosure provides a light-emitting marker comprising a light-emitting core comprising a light-emitting material; a first group bound to the light-emitting core and comprising a first biotin group; a second biotin group bound to a biomolecule, and a protein bound to the first and second biotin groups.

The light-emitting marker may be as described herein. The light-emitting marker may comprise a core, a first group, a second group, a protein group, a first surface group, a second surface group or a matrix as described anywhere herein.

In some embodiments, there is provided a colloid containing light-emitting marker particles as described herein suspended in a liquid. Optionally, the liquid comprises or consists of water. Optionally, the liquid is a buffer solution.

In some embodiments, there is provided a method of forming a light-emitting marker as described herein, in which the biomolecule bound to the second biotin group is contacted with a precursor light-emitting marker having the first biotin group and the protein bound to the first biotin group. Optionally, following contacting of these materials and before the light-emitting marker is used in an assay, any biomolecule bound to the second biotin group which has not bound to the precursor light-emitting marker is separated from the light-emitting marker.

### DESCRIPTION OF THE DRAWINGS

The present disclosure is described in conjunction with the appended figures. It is emphasized that, in accordance with the standard practice in the industry, various features are not drawn to scale. In fact, the dimensions of the various features may be arbitrarily increased or reduced for clarity of discussion.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

The invention will now be described in more detail with reference to the drawings wherein:

FIG. 1 is a schematic illustration of a nanoparticle according to some embodiments;

FIG. 2 is schematic illustration of a method of forming a nanoparticle according to some embodiments;

FIG. 3 is a reaction scheme for forming a DBCO-functionalised antibody;

FIG. 4 is a bar chart of a plate assay using light-emitting nanoparticles conjugated to a biotinylated antibody according to an embodiment of the present disclosure;

FIG. 5 is a bar chart of a plate assay using comparative light-emitting nanoparticles in which antibodies are conjugated to the nanoparticles by EDC/NHS chemistry;

FIG. 6 is a bar chart of a plate assay using comparative light-emitting nanoparticles in which antibodies are conjugated to the nanoparticles by azide/alkyne click chemistry;

FIG. 7 is a staining index chart for Cyto-Trol cells stained with a biotinylated dissolved light-emitting polymer marker according to an embodiment of the present disclosure conjugated through streptavidin to anti-human CD4 antibody; and a comparative light-emitting nanoparticle conjugated to the same antibody.

FIG. 8 is a staining index chart for Cyto-Trol cells stained with the streptavidin-conjugated nanoparticle of FIG. 7; a light-emitting nanoparticle conjugated to an isotype; and a light-emitting nanoparticle which is not conjugated to streptavidin.

FIG. 9 is a staining index chart for Cyto-Trol cells stained with a biotinylated nanoparticle according to an embodiment of the present disclosure conjugated through neutravidin to anti-human CD4 antibody; and a comparative dissolved light-emitting polymer marker conjugated to the same antibody.

FIG. 10 is a staining index chart for Cyto-Trol cells stained with the neutravidin-conjugated nanoparticle of FIG. 9; a light-emitting nanoparticle conjugated to an isotype; and a light-emitting nanoparticle which is not conjugated to neutravidin.

FIG. 11 is a staining index chart for Cyto-Trol cells stained with biotinylated nanoparticles having differing amounts of biotin according to embodiments of the present disclosure conjugated through streptavidin to anti-human CD4 antibody; and a comparative dissolved light-emitting polymer marker conjugated to the same antibody.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements. Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

The teachings of the technology provided herein can be applied to other systems, not necessarily the system described below. The elements and acts of the various examples described below can be combined to provide further implementations of the technology.

Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have found that a biotinylated light-emitting core which is bound to a biotinylated biomolecule such as an antibody through a protein having a plurality of biotin binding sites, e.g. streptavidin, can give assays with a high signal-to-noise ratio, low standard deviation and/or in the case of flow cytometry, a high staining index.

A wide range of biotinylated antibodies are commercially available and so light-emitting markers suitable for detecting a correspondingly wide range of antigens may be formed from a protein-conjugated biotinylated light-emitting cores.

Preferably, the binding between the biotinylated biomolecule and the protein occurs upon mixing at ambient temperature, e.g. 20° C., or at a lower temperature, e.g. in the range of 0-20° C., i.e. without the need for any activation such as in EDC/NHS binding.

Any excess of the biotinylated biomolecule which remains unbound, via the protein, to the biotinylated light-emitting core may be separated from the light-emitting marker.

In some embodiments, the light-emitting core of the light-emitting marker may consist of an organic light-emitting material, e.g. a non-polymeric organic light-emitting compound or a light-emitting polymer. The first biotin group of the light-emitting marker according to these embodiments may be bound directly to the light-emitting material, or bound through a binding group disposed between the light-emitting material and the biotin group. In use, the light-emitting marker may be dissolved or dispersed in a carrier liquid.

The light-emitting core may be a light-emitting particle having a particulate light-emitting core comprising or consisting of an organic or inorganic light-emitting material. The core may comprise a matrix material mixed with or bound to the light-emitting material. In use, the light-emitting particle is dispersed in a carrier liquid. The light-emitting particle core may comprise plural copies of a light-emitting material, which may result in higher brightness of the light-emitting marker as compared to a light-emitting marker containing a single light-emitting material. The first biotin group of the light-emitting marker according to these embodiments may be bound to the light-emitting material or to the matrix material. The first biotin group may be bound directly to the light-emitting material or the matrix material or may be bound through a binding group disposed between the light-emitting material and the biotin group.

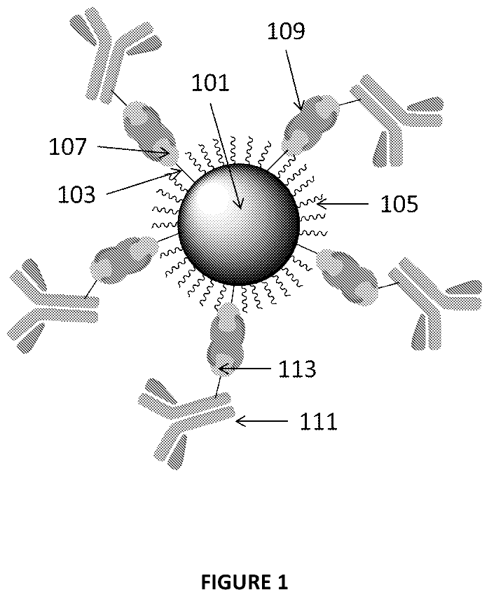

FIG. 1 illustrates a particle 100 according to some embodiments of the present disclosure.

The light-emitting particle 100 has a core 101 comprising or consisting of a light-emitting material. Preferably, the core comprises more than one molecule of the light-emitting material, e.g. more than one light-emitting polymer chain in the case of a light-emitting polymer material.

The core may comprise a host material and a chromophore wherein the host material is configured to absorb excitation energy from an energy source, e.g. a light source, and transfer energy to the chromophore, and wherein the chromophore is configured to emit light upon transfer of energy from the host material. Each of the host material and chromophore is independently a non-polymeric or polymeric material.

The core 101 may comprise a matrix material, optionally a polymeric or inorganic matrix material. Exemplary polymeric matrix materials include, without limitation, polystyrene and homopolymers or copolymers of (alkyl)acrylic acids. A polymeric matrix material may be crosslinked, e.g. a crosslinked chitosan-polyacrylic acid polymer. The polymer matrix may be a self-assembled micelle or vesicle comprising lipid or polymer surfactants. The polymer matrix is preferably an inorganic oxide. The polymer matrix is more preferably silica.

If a matrix material is present then in some embodiments the light-emitting material may be covalently bound, directly or indirectly, to the matrix material. In some embodiments, the light-emitting material may be mixed with (i.e. not covalently bound to) a matrix material. The light-emitting material may be distributed homogenously or non-homogeneously in the light-emitting material/matrix mixture.

The core 101 may contain one or more light-emitting polymer chains mixed with and extending through the matrix material. One or more light-emitting polymer chains may protrude beyond a surface of the core defined by the matrix material.

The core 101 may comprise a shell, e.g. a silica shell, partially or completely covering an inner core comprising or consisting of a light-emitting material, preferably a light-emitting polymer.

Preferably, the particles have a number average diameter of no more than 5000 nm, more preferably no more than 2500 nm, 1000 nm, 900 nm, 800 nm, 700 nm, 600 nm, 500 nm or 400 nm as measured by dynamic light scattering (DLS) using a Malvern Zetasizer Nano ZS. Preferably the particles have a number average diameter of between 5-5000 nm, optionally 10-1000 nm, preferably between 10-500 nm, most preferably between 10-100 nm as measured by a Malvern Zetasizer Nano ZS.

In some embodiments, the light-emitting material is inorganic. Optionally, the inorganic light-emitting material is a quantum dot.

In some embodiments, the light-emitting material is organic. In some embodiments, the organic light-emitting material is non-polymeric. In some embodiments, the organic light-emitting material is a light-emitting polymer.

A first surface group 103 is bound to the light-emitting particle core. Optionally, one or more further surface groups are also bound to the light-emitting particle core, e.g. second surface group 105. In some embodiments, the first surface group 103 and/or second surface group 105 may be covalently bound to the light-emitting material of the light-emitting particle core. Preferably, the first surface group 103 and/or second surface group 105 are covalently bound to a matrix material of the light-emitting particle core.

The first surface group 103 comprises a first biotin group 107. The second surface group 105 does not comprise biotin.

The first biotin group may be bound to the surface of the light-emitting particle core by a biotin binding group which is bound at one end to the core and at another end to the biotin.

The first biotin group is bound to a protein 109 having a plurality of biotin binding sites, preferably streptavidin, neutravidin, avidin or a recombinant variant or derivative thereof. Preferably, the protein is not luminescent. A biotinylated biomolecule 111, e.g. a biotinylated IgG antibody as illustrated in FIG. 1, having a second biotin group 113 is bound to the same protein.

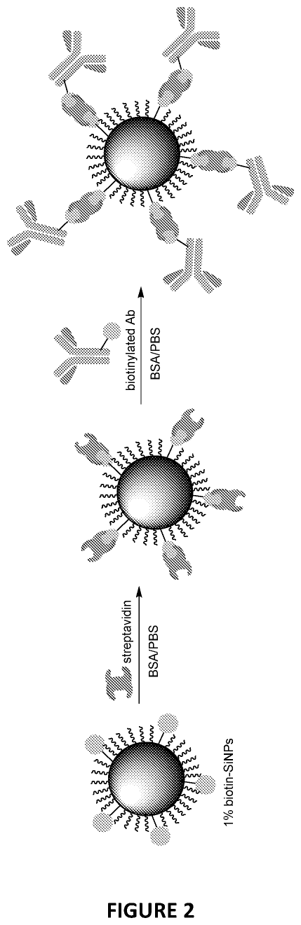

FIG. 2 illustrates a process of forming light-emitting marker particles as described herein according to some embodiments of the present disclosure.

A light-emitting particle core comprising or consisting of a light-emitting material may be formed by any process described herein. Preferably, silica is disposed at the surface of the light-emitting particle core. A reactive group RG1 may be disposed at the surface of the light-emitting particle core.

A first material having a reactive group RG2 may be brought into contact with the light-emitting particle core under conditions for reaction between RG1 and RG2, thereby binding the first material to the light-emitting particle core. The first material may comprise biotin or may comprise a third reactive group RG3 for binding to biotin following reaction between RG1 and RG2.

In addition to the first material, a second material having a reactive group RG2 may be brought into contact with the light-emitting particle core wherein RG2 of the first and second materials may be the same or different. The first and second materials may be brought into contact with the particle core sequentially in either order or simultaneously.

The second material either does not comprise biotin or does not have a group capable of binding to biotin. The ratio of the first and second materials brought into contact with the light-emitting particle core may be selected to control the amount of biotin of the light-emitting marker particle and, therefore, the number of proteins and biotinylated antibodies capable of binding to the light-emitting nanoparticle.

Preferably, the number of second surface groups 105 is greater than the number of first surface groups 103. Optionally, the number of moles of the second surface groups is at least 2 times, preferably 3 times, more preferably at least 5 times, the number of moles of the first surface groups. Most preferably, the number of first surface groups is less than 10 mol %, optionally up to 5 mol %, of the total number of moles of the first and second surface groups.

Preferably, the number of first surface groups is more than 0.1 mol %, optionally at least 0.5 mol %, of the total number of moles of the first and second surface groups.

Biotin of the first surface groups may be conjugated to a protein having more than one biotin binding site to form a precursor particle to which a biotinylated biomolecule may be conjugated. The protein could be native, e.g. native streptavidin. The protein could be recombinant, e.g. divalent streptavidin.

The biomolecule may be, without limitation, an antibody; an antigen-binding fragment (Fab); a mimetic, e.g. a minibody, nanobody, monobody, diabody or triabody or affibody; a DARPin; or a fusion protein, e.g. a single-chain variable fragment (scFv); a linear or cyclic peptide; annexin V; RNA or DNA; or an aptamer.

An antibody biomolecule may be selected according to the antigen to be detected. A wide range of biotinylated antibodies are known and commercially available, or may be prepared using techniques known the skilled person, e.g. as disclosed in, for example, https://www.abcam.com/ps/pdf/protocols/biotin_conjugation.pdf, the contents of which are incorporated herein by reference.

The second surface group 105 may be selected from groups described anywhere herein with reference to the first surface group except that it does not comprise biotin. The second surface group 105 may be as described with reference to the first surface group wherein the first biotin group (and the protein and biotinylated antibody) is replaced with another group including, without limitation, H; C₁₋₁₂ alkyl; C₁₋₁₂ alkoxy; OH; —N(R⁵)₂ wherein each R⁵ is independently H or C₁₋₁₂ hydrocarbyl; COOH; and esters of COOH, e.g. C₁₋₂₀ hydrocarbyl esters of COOH. The first and second groups of a light-emitting marker particle may differ only in the presence or absence of the protein and the biotin groups bound thereto, or may differ in one or more further respects.

Optionally, the second surface group does not bind to an antigen of the antibody of the first surface group when brought into contact with the antigen in water at 25° C.

The first and second surface groups may be polydisperse. The second surface group may have a Mn of at least 500, optionally at least 2,000. The first surface group, not including the first and second biotin groups, antibody and protein, may have a Mn of at least 500, optionally at least 2,000.

The first and second surface group may each independently have a multimodal weight distribution, optionally a bimodal weight distribution. A multimodal weight distribution may be achieved by mixing polydisperse materials having different average molecular weights.

Formation of First and Further Surface Groups

In some embodiments, the first surface group may be formed by reaction of a first material of formula (Ia):

RG2-PG-Biotin (Ia)

in which PG is a polar group and RG2 reacts to bind to a group RG1 on the surface of the light-emitting particle core.

The first surface group may be formed by reaction of a first material of formula (Ib):

RG2-PG-RG3 (I)

in which PG is a polar group; RG2 reacts to bind to a group RG1 on the surface of the light-emitting particle core and RG3 is capable of binding to biotin. Following reaction between RG1 and RG2, RG3 may be bound to biotin.

PG may be a linear or branched polar group.

PG may comprise heteroatoms capable of forming hydrogen bonds with water, optionally a linear or branched alkylene chain wherein one or more C atoms of the alkylene chain are replaced with 0 or NR⁶ wherein R⁶ is a C₁₋₁₂ hydrocarbyl group, optionally a C₁₋₁₂ alkyl group or C₁₋₄ alkyl group.

Preferably, PG has a molecular weight of less than 5,000, optionally in the range of 130-3500 Da.

Preferably, PG is a polyether chain. By “polyether chain” as used herein is meant a divalent chain comprising a plurality of ether groups.

Preferably, PG comprises a group of formula (II):

—((CR¹⁴R¹⁵)_{b}O)_{c}— (II)

wherein R¹⁴ and R¹⁵ are each independently H or C₁₋₆ alkyl and b is at least 1, optionally 1-5, preferably 2, and c is at least 2, optionally 2-1,000, preferably 10-500, 10-200 or 10-100, most preferably 10-50.

Most preferably, PG comprises or consists of a polyethylene glycol chain.

RG1 and RG2 may be selected from:

amine groups, optionally —NR⁸₂ wherein R⁸ in each occurrence is independently H or a substituent, preferably H or a C₁₋₅ alkyl, more preferably H;

carboxylic acid or a derivative thereof which forms a carboxylic acid group or a salt thereof in the reaction between RG1 and RG2, for example an anhydride, acid chloride or ester;

—OH; —SH; an alkene; an alkyne; and an azide.

RG1 and RG2 may react to form a group selected from esters, amides, urea, thiourea, Schiff bases, a primary amine (C—N) bond, a maleimide-thiol adduct or a triazole formed by the cycloaddition of an azide and an alkyne.

Preferably, one of RG1 and RG2 is a carboxylic acid or a derivative thereof such as an ester, preferably an NHS ester, acid chloride or acid anhydride group and the other of RG1 and RG2 is a protic group such as a hydroxyl, thiol or amino group, preferably an amino group, wherein RG1 and RG2 are capable of reaction to form an ester or amide group. One of RG1 and RG2 may be converted to an activated form before reaction, for example activation of a carboxylic acid group using a carbodiimide, for example EDC.

Optionally, the reactive group RG1 is formed at the surface of the light-emitting particle core by reacting a compound comprising RG1 with the particle core.

Optionally, the particle core comprises silica and the compound comprising reactive group RG1 has formula (II):

(R⁷O)₃Si—(Sp¹)ₓ-RG1 (II)

wherein R⁷ is H or a substituent, preferably a C₁₋₁₀ alkyl group;

Sp¹ is a spacer group;

x is 0 or 1; and

RG1 is a first reactive group.

Preferably, a silane formed by reaction of the compound of formula (I) forms a monolayer on silica at the surface of the particle core.

Optionally, Sp¹ is selected from a linear or branched divalent alkylene chain wherein one or more non-adjacent C atoms may be replaced with O, S, C(═O), C(═O)O, C(═O)NR¹² or NR¹², wherein R¹² in each occurrence is independently selected from H and C₁₋₁₂ hydrocarbyl, optionally C₁₋₁₂ alkyl.

An exemplary compound of formula (II) is 3-aminopropyl triethoxysilane.

Second surface group 105 may be as described for first surface group RG1 except for the presence of biotin.

Particle Core

The particle core may consist of one or more light-emitting materials.

The particle core may comprise or consist of one or more light-emitting materials and a matrix material. Matrix materials include, without limitation, inorganic matrix materials, optionally inorganic oxides, optionally silica.

In some embodiments, the particle core may be formed by polymerisation of a silica monomer in the presence of a light-emitting material, for example as described in WO 2018/060722, the contents of which are incorporated herein by reference.

In some embodiments, the particle core comprises an inner core which comprises or consists of at least one light-emitting material and at least one shell surrounding the inner core. The at least one shell may be silica.

Optionally, at least 0.1 wt % of total weight of the particle core consists of one or more light-emitting materials. Preferably at least 1, 10, 25 wt % of the total weight of the particle core consists of one or more light-emitting materials.

Optionally at least 50 wt % of the total weight of the particle core consists of the silica. Preferably at least 60, 70, 80, 90, 95, 98, 99, 99.5, 99.9 wt % of the total weight of the particle core consists of silica.

The particle core as described herein is the light-emitting particle without any surface groups thereon.

In one embodiment of the present disclosure, at least 70 wt % of the total weight of the particle core consists of the light-emitting material or materials and silica. Preferably at least 80, 90, 95, 98, 99, 99.5, 99.9 wt % of the total weight of the particle core consists of the light-emitting material or materials and silica. More preferably the particle core consists essentially of the one or more light-emitting materials and silica.

Light-Emitting Materials

The light-emitting core of the light-emitting marker may comprise or consist of a light-emitting material which emits fluorescent light, phosphorescent light or a combination thereof.

The light-emitting material may emit light having a peak wavelength in the range of 350-1000 nm.

A blue light-emitting material as described herein may have a photoluminescence spectrum with a peak of no more than 500 nm, preferably in the range of 400-500 nm, optionally 400-490 nm.

A green light-emitting material as described herein may have a photoluminescence spectrum with a peak of more than 500 nm up to 580 nm, optionally more than 500 nm up to 540 nm.

A red light-emitting material as described herein may have a photoluminescence spectrum with a peak of no more than more than 580 nm up to 950 nm, optionally up to 630 nm, optionally 585 nm up to 625 nm.

The light-emitting material may have a Stokes shift in the range of 10-850 nm.

UV/vis absorption spectra of light-emitting markers as described herein may be as measured in methanol solution or suspension using a Cary 5000 UV-vis-IR spectrometer.

Photoluminescence spectra of light-emitting particles as described herein may be measured in methanol solution or suspension using a Jobin Yvon Horiba Fluoromax-3.

The light-emitting material may be an inorganic light-emitting material; a non-polymeric organic light-emitting material; or a light-emitting polymer.

Exemplary non-polymeric fluorescent materials include, without limitation: fluorescein and salts thereof, for example, fluorescein isothiocyanate (FITC), fluorescein NHS, Alexa Fluor 488, Dylight 488, Oregon green, DAF-FM, 6-FAM2,7-dichlorofluorescein, 3′-(p-aminophenyl)fluorescein and 3′-(hydroxyphenyl)fluorescein; rhodamines, for example Rhodamine 6G and Rhodamine 110 chloride; coumarins; boron-dipyrromethenes (BODIPYs); naphthalimides; perylenes; benzanthrones; benzoxanthrones; and benzothiooxanthrones, each of which may be unsubstituted or substituted with one or more substituents. Exemplary substituents are chlorine, alkyl amino; phenylamino; and hydroxyphenyl.

Light-emitting polymers are preferred.

The light-emitting polymer may be a homopolymer or may be a copolymer comprising two or more different repeat units.

The light-emitting polymer may comprise light-emitting groups in the polymer backbone, pendant from the polymer backbone or as end groups of the polymer backbone. In the case of a phosphorescent polymer, a phosphorescent metal complex, preferably a phosphorescent iridium complex, may be provided in the polymer backbone, pendant from the polymer backbone or as an end group of the polymer backbone.

The light-emitting polymer may have a non-conjugated backbone or may be a conjugated polymer. By “conjugated polymer” is meant a polymer comprising repeat units in the polymer backbone that are directly conjugated to adjacent repeat units. Conjugated light-emitting polymers include, without limitation, polymers comprising one or more of arylene, heteroarylene and vinylene groups conjugated to one another along the polymer backbone.

The light-emitting polymer may have a linear, branched or crosslinked backbone.

The light-emitting polymer may comprise one or more repeat units in the backbone of the polymer substituted with one or more substituents selected from non-polar and polar substituents.

Preferably, the light-emitting polymer comprises at least one polar substituent. The one or more polar substituents may be the only substituents of said repeat units, or said repeat units may be further substituted with one or more non-polar substituents, optionally one or more C₁₋₄₀ hydrocarbyl groups. The repeat unit or repeat units substituted with one or more polar substituents may be the only repeat units of the polymer or the polymer may comprise one or more further co-repeat units wherein the or each co-repeat unit is unsubstituted or is substituted with non-polar substituents, optionally one or more C₁₋₄₀ hydrocarbyl substituents.

C₁₋₄₀ hydrocarbyl substituents as described herein include, without limitation, C₁₋₂₀ alkyl, unsubstituted phenyl and phenyl substituted with one or more C₁₋₂₀ alkyl groups.

As used herein a “polar substituent” may refer to a substituent, alone or in combination with one or more further polar substituents, which renders the light-emitting polymer with a solubility of at least 0.01 mg/ml in an alcoholic solvent, optionally in the range of 0.01-10 mg/ml. Optionally, solubility is at least 0.1 or 1 mg/ml. The solubility is measured at 25° C. Preferably, the alcoholic solvent is a C₁₋₁₀ alcohol, more preferably methanol.

Polar substituents are preferably substituents capable of forming hydrogen bonds or ionic groups.

In some embodiments, the light-emitting polymer comprises polar substituents of formula —O(R³O)ₜ—R⁴ wherein R³ in each occurrence is a C₁₋₁₀ alkylene group, optionally a C₁₋₅ alkylene group, wherein one or more non-adjacent, non-terminal C atoms of the alkylene group may be replaced with 0, R⁴ is H or C₁₋₅ alkyl, and t is at least 1, optionally 1-10. Preferably, t is at least 2. More preferably, t is 2 to 5. The value of t may be the same in all the polar groups of formula —O(R³O)ₜ—R⁴. The value of t may differ between polar groups of the same polymer.

By “C₁₋₅ alkylene group” as used herein with respect to R³ is meant a group of formula —(CH₂)_{f}— wherein f is from 1-5.

Preferably, the light-emitting polymer comprises polar substituents of formula —O(CH₂CH₂O)ₜ—R⁴ wherein t is at least 1, optionally 1-10 and R⁴ is a C₁₋₅ alkyl group, preferably methyl. Preferably, t is at least 2. More preferably, t is 2 to 5, most preferably q is 3.

In some embodiments, the light-emitting polymer comprises polar substituents of formula —N(R⁵)₂, wherein R⁵ is H or C₁₋₁₂ hydrocarbyl. Preferably, each R⁵ is a C₁₋₁₂ hydrocarbyl.

In some embodiments, the light-emitting polymer comprises polar substituents which are ionic groups which may be anionic, cationic or zwitterionic. Preferably the ionic group is an anionic group.

Exemplary anionic groups are —COO⁻, a sulfonate group; hydroxide; sulfate; phosphate; phosphinate; or phosphonate.

An exemplary cationic group is —N(R⁵)₃⁺ wherein R⁵ in each occurrence is H or C₁₋₁₂ hydrocarbyl. Preferably, each R⁵ is a C₁₋₁₂ hydrocarbyl.

A light-emitting polymer comprising cationic or anionic groups comprises counterions to balance the charge of these ionic groups.

An anionic or cationic group and counterion may have the same valency, with a counterion balancing the charge of each anionic or cationic group.

The anionic or cationic group may be monovalent or polyvalent. Preferably, the anionic and cationic groups are monovalent.

The light-emitting polymer may comprise a plurality of anionic or cationic polar substituents wherein the charge of two or more anionic or cationic groups is balanced by a single counterion. Optionally, the polar substituents comprise anionic or cationic groups comprising di- or trivalent counterions.

The counterion is optionally a cation, optionally a metal cation, optionally Li⁺, Na⁺, K⁺, Cs⁺, preferably Cs⁺, or an organic cation, optionally ammonium, such as tetraalkylammonium, ethylmethyl imidazolium or pyridinium.

The counterion is optionally an anion, optionally a halide; a sulfonate group, optionally mesylate or tosylate; hydroxide; carboxylate; sulfate; phosphate; phosphinate; phosphonate; or borate.

In some embodiments, the light-emitting polymer comprises polar substituents selected from groups of formula —O(R³O)ₜ—R⁴, groups of formula —N(R⁵)₂, groups of formula OR⁴ and/or ionic groups. Preferably, the light-emitting polymer comprises polar substituents selected from groups of formula —O(CH₂CH₂O)ₜR⁴, groups of formula —N(R⁵)₂, and/or anionic groups of formula —COO⁻. Preferably, the polar substituents are selected from the group consisting of groups of formula —O(R³O)ₜ—R⁴, groups of formula —N(R⁵)₂, and/or ionic groups. Preferably, the polar substituents are selected from the group consisting of polyethylene glycol (PEG) groups of formula —O(CH₂CH₂O)ₜR⁴, groups of formula —N(R⁵)₂, and/or anionic groups of formula —COO⁻. R³, R⁴, R⁵, and t are as described above.

Optionally, the backbone of the light-emitting polymer is a conjugated polymer. Optionally, the backbone of the conjugated light-emitting polymer comprises repeat units of formula (III):

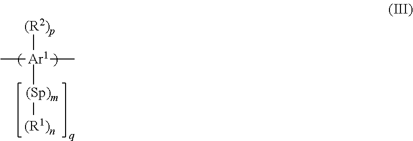

wherein Ar¹ is an arylene group or heteroarylene group; Sp is a spacer group; m is 0 or 1; R¹ independently in each occurrence is a polar substituent; n is 1 if m is 0 and n is at least 1, optionally 1, 2, 3 or 4, if m is 1; R² independently in each occurrence is a non-polar substituent; p is 0 or a positive integer, optionally 1, 2, 3 or 4; q is 0 or a positive integer, optionally 1, 2, 3 or 4; and wherein Sp, R¹ and R² may independently in each occurrence be the same or different. Two substituents of Ar¹ may be linked to form a ring.

Preferably, m is 1 and n is 2-4, more preferably 4.

Preferably p is 0.

Preferably q is at least 1.

Ar¹ of formula (III) is optionally a C₆₋₂₀ arylene group or a 5-20 membered heteroarylene group. Ar¹ is preferably a dibenzosilole group or a C₆₋₂₀ arylene group, optionally phenylene, fluorene, benzofluorene, phenanthrene, naphthalene or anthracene, more preferably fluorene or phenylene, most preferably fluorene.

Exemplary Ar¹ groups of formula (III) include groups of formula (IV)-(X):

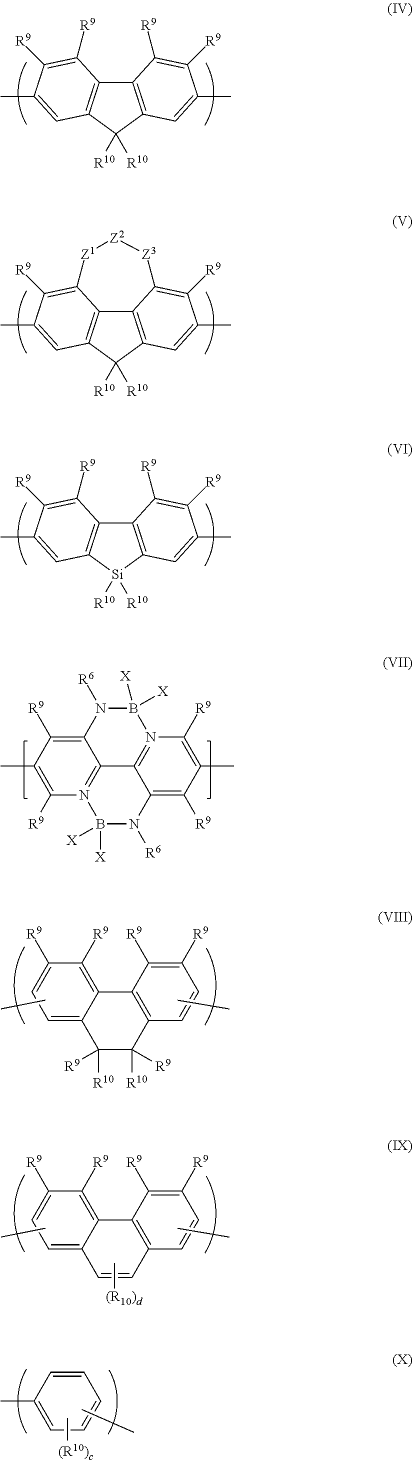

wherein

R⁹ in each occurrence is independently H, R¹ or R², preferably H;

R¹⁰ in each occurrence is independently R¹ or R²; R⁶ is a C₁₋₁₂ hydrocarbyl group, optionally a C₁₋₁₂ alkyl group or C₁₋₄ alkyl group;

c is 0, 1, 2, 3 or 4, preferably 1 or 2;

d is 0, 1 or 2;

X independently in each occurrence is a substituent, preferably a substituent selected from the group consisting of branched, linear or cyclic C₁₋₂₀ alkyl; phenyl which is unsubstituted or substituted with one or more substituents, e.g. one or more C₁₋₁₂ alkyl groups; and F; and

Z¹-Z²-Z³ is a C₂ (ethylene) C₃ alkylene (propylene) chain wherein one or two non-adjacent C atoms may be replaced with O, S or NR⁶.

Sp-(R¹)n may be a branched group, optionally a dendritic group, substituted with polar groups, optionally —NH₂ or —OH groups, for example polyethyleneimine.

Preferably, Sp is selected from:
C₁₋₂₀ alkylene or phenylene-C₁₋₂₀ alkylene wherein one or more non-adjacent C atoms may be replace with O, S, N or C═O;
a C₆₋₂₀ arylene or 5-20 membered heteroarylene, more preferably phenylene, which, in addition to the one or more substituents R¹, may be unsubstituted or substituted with one or more non-polar substituents, optionally one or more C₁₋₂₀ alkyl groups.

“alkylene” as used herein means a branched or linear divalent alkyl chain.

“non-terminal C atom” of an alkyl group as used herein means a C atom other than the methyl group at the end of an n-alkyl group or the methyl groups at the ends of a branched alkyl chain.

More preferably, Sp is selected from:
C₁₋₂₀ alkylene wherein one or more non-adjacent C atoms may be replaced with O, S or CO; and
a C₆₋₂₀ arylene or a 5-20 membered heteroarylene, even more preferably phenylene, which may be unsubstituted or substituted with one or more non-polar substituents.

R¹ may be a polar substituent as described anywhere herein. Preferably, R¹ is:
a polyethylene glycol (PEG) group of formula —O(CH₂CH₂O)ₜR⁴ wherein t is at least 1, optionally 1-10 and R⁴ is a C₁₋₅ alkyl group, preferably methyl;
a group of formula —N(R⁵)₂, wherein R⁵ is H or C₁₋₁₂ hydrocarbyl; or
an anionic group of formula —COO⁻.

In the case where n is at least two, each R¹ may independently in each occurrence be the same or different. Preferably, each R¹ attached to a given Sp group is different.

In the case where p is a positive integer, optionally 1, 2, 3 or 4, the group R² may be selected from:
alkyl, optionally C₁₋₂₀ alkyl; and
aryl and heteroaryl groups that may be unsubstituted or substituted with one or more substituents, preferably phenyl substituted with one or more C₁₋₂₀ alkyl groups;
a linear or branched chain of aryl or heteroaryl groups, each of which groups may independently be substituted, for example a group of formula —(Ar³)ₛ wherein each Ar³ is independently an aryl or heteroaryl group and s is at least 2, preferably a branched or linear chain of phenyl groups each of which may be unsubstituted or substituted with one or more C₁₋₂₀ alkyl groups; and
a crosslinkable-group, for example a group comprising a double bond such and a vinyl or acrylate group, or a benzocyclobutane group.

Preferably, each R², where present, is independently selected from C₁₋₄₀ hydrocarbyl, and is more preferably selected from C₁₋₂₀ alkyl; unsubstituted phenyl; phenyl substituted with one or more C₁₋₂₀ alkyl groups; and a linear or branched chain of phenyl groups, wherein each phenyl may be unsubstituted or substituted with one or more substituents.

A polymer as described herein may comprise or consist of only one form of the repeating unit of formula (III) or may comprise or consist of two or more different repeat units of formula (III).

Optionally, the polymer comprising one or more repeat units of formula (III) is a copolymer comprising one or more co-repeat units.

If co-repeat units are present then the repeat units of formula (III) may form between 0.1-99 mol % of the repeat units of the polymer, optionally 50-99 mol % or 80-99 mol %. Preferably, the repeat units of formula (I) form at least 50 mol % of the repeat units of the polymer, more preferably at least 60, 70, 80, 90, 95, 98 or 99 mol %. Most preferably the repeat units of the polymer consist of one or more repeat units of formula (I).

The or each repeat unit of the polymer may be selected to produce a desired colour of emission of the polymer.

Arylene repeat units of the polymer include, without limitation, fluorene, preferably a 2,7-linked fluorene; phenylene, preferably a 1,4-linked phenylene; naphthalene, anthracene, indenofluorene, phenanthrene and dihydrophenanthrene repeat units.

The polystyrene-equivalent number-average molecular weight (Mn) measured by gel permeation chromatography of the light-emitting polymers or the silica polymers described herein may be in the range of about 1×10³ to 1×10⁸, and preferably 1×10⁴ to 5×10⁶. The polystyrene-equivalent weight-average molecular weight (Mw) of the polymers described herein may be 1×10³ to 1×10⁸, and preferably 1×10⁴ to 1×10⁷.

Polymers as described herein are suitably amorphous polymers.

Colloids

The particles may be provided as a colloidal suspension comprising the particles suspended in a liquid. Preferably, the liquid is selected from water, C₁₋₁₀ alcohols and mixtures thereof. Preferably, the particles form a uniform (non-aggregated) colloid in the liquid.

The liquid may be a solution comprising salts dissolved therein, optionally a buffer solution. The buffer solution may have a pH in the range of 1-14, preferably 5-8. The buffer solution may contain without limitation, a phosphate e.g. sodium phosphate, tris(hydroxymethyl)aminomethane (tris), an acetate e.g. sodium acetate, a borate, and/or 2-(N-morpholino)ethanesulfonic acid (MES).

The salt concentration of a buffer solution may be in the range of about 1 mmol/L-200 mmol/L.

The concentration of the particles in the colloidal suspension is preferably in the range of 0.1-20 mg/mL, optionally 5-20 mg/mL.

In some embodiments, the particles may be stored as a colloidal suspension, optionally a colloidal suspension having a particle concentration greater than 0.1 mg/mL, preferably at least 0.5 mg/mL or 1 mg/mL.

In some embodiments, the particles may be stored in a lyophilised or frozen form.

Applications

The particles of the present disclosure may be fluorescent or phosphorescent. Preferably the particles are fluorescent. Preferably the particles are for use as a luminescent probe, e.g. a fluorescent probe for detecting a biomolecule or for labelling a biomolecule. In some embodiments, the particles may be used as a luminescent probe, e.g. a fluorescent probe in an immunoassay such as a lateral flow or solid state immunoassay. Optionally the particles are for use in fluorescence microscopy, flow cytometry, next generation sequencing, in-vivo imaging, or any other application where a light-emitting marker configured to bind to a target analyte is brought into contact with a sample to be analysed. The applications can medical, veterinary, agricultural or environmental applications whether involving patients (where applicable) or for research purposes.

Preferably, the presence and/or concentration of a target analyte comprises measurement of any light-emitting markers dispersed or dissolved in the sample which are bound to the target analyte (as opposed to light-emitting markers bound to the target analyte and immobilised on a surface).

Preferably, the presence and/or concentration of a target analyte comprises detection of light emitted directly from the light emitting marker.

In some embodiments, a sample to be analysed may brought into contact with the particles, for example the particles in a colloidal suspension.

In some embodiments, the sample following contact with the particles is analysed by flow cytometry. In flow cytometry, the particles are irradiated by at least one wavelength of light, optionally two or more different wavelengths, e.g. one or more wavelengths including at least one of 355, 405, 488, 562 and 640 nm. Light emitted by the particles may be collected by one or more detectors. Detectors may be selected from, without limitation, photomultiplier tubes and photodiodes. To provide a background signal for calculation of a staining index, measurement may be made of particles mixed with cells which do not bind to the particles.

In some embodiments, e.g. a plate assay, any target antigen in the sample may be immobilised on a surface which is brought into contact with the particles.

### Example 1—Streptavidin-Functionalised Precursor Nanoparticle Formation

Formation of Light-Emitting Nanoparticle Nuclei with Reactive Amine Groups

Nanoparticles having a core of silica and a light-emitting polymer were formed by the Stöber process and the nanoparticle nuclei were reacted with (3-aminopropyl)triethoxysilane as described in the examples of WO 2018/060722, the contents of which are incorporated herein by reference, to give nanoparticles with a number average diameter by dynamic light scattering of 80 nm and amine reactive groups on the surface of the nuclei.

Attachment of Surface Groups to Amino-Modified Light-Emitting Nanoparticles

1 mL of the suspension of amino-modified nanoparticle nuclei in methanol formed in the example above was centrifuged at 14,000 rpm for 2 minutes to isolate the nanoparticles through decantation of the supernatant. A 1 mL solution of 9.9 mg α,ω-Bis{2-[(3-carboxy-1-oxopropyl)amino]ethyl}polyethylene glycol (SAA-PEG-SAA, illustrated below, MW=2000 g/mol), 0.1 mg biotin-PEG-COOH (MW=2000 g/mol), N-(3-aminopropyl)-N-ethylcarbodiimide (2.1 mg) and N-hydroxysuccinimide (2.5 mg) in methanol was used to redisperse 5 mg of the nanoparticle pellet by gentle sonication and the resultant suspension was stirred at room temperature for 1 hour.

The suspension was centrifuged at 14,000 rpm for 2 minutes to isolate the resultant silica-LEP nanoparticles from the supernatant containing excess unreacted PEGylation reagents. The supernatant was removed by decantation and gentle sonication was used to redisperse the isolated pellet of nanoparticles in 1 mL of fresh methanol. Wash cycles consisting of centrifugation, decantation and redispersion in methanol (1 mL) were repeated a further two times.

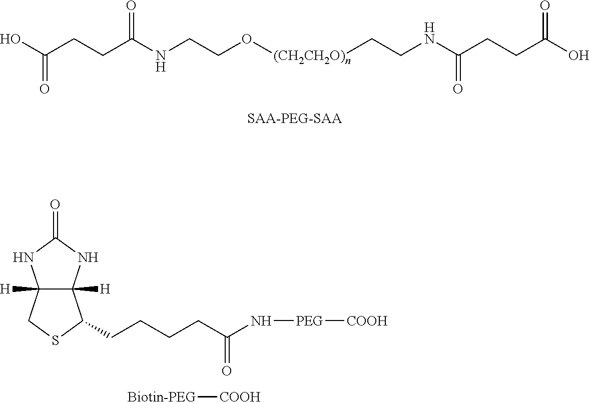

One of the isolated PEGylated nanoparticle pellets was resuspended in 1 mL of phosphate buffered saline (pH 7.4, containing 1 wt. % bovine serum albumin) by gentle sonication, followed by immediate addition of 50 μL of a solution of streptavidin in the same buffer (1 mg/mL). The suspension was stirred at room temperature for 1 hour before centrifuging the sample at 14000 rpm for 3 minutes to collect separate the protein-conjugated nanoparticles from the supernatant and unconjugated protein. The pellet was resuspended by gentle sonication in 100 μL of phosphate buffered saline with 1% BSA for storage.

### Example 2—Conjugation to Biotinylated Antibody

To 1 mg of the streptavidin-functionalised nanoparticles described in Example 1, 250 μL of 0.5 mg/mL biotinylated goat anti-mouse antibody (clone Poly4053, purchased from Biolegend) was added, and the mixture was agitated for 1 h at room temperature. This step was repeated four times; on the final step the pellet was resuspended in 500 μL BSA/PBS to give a final particle concentration of 2 mg/mL.

### Comparative Example 1—EDC/NHS Conjugated Particles

For the purpose of comparison, nanoparticles having a surface group carrying NHS ester rather than biotin were conjugated to an antibody.

Light-emitting nanoparticle nuclei with reactive amine groups as described in Example 1, i.e. before attachment of surface groups as described in Example 1, were reacted with succinic anhydride to form a carboxyl group at the particle core surface and functionalised by activating the carboxyl group using EDC and sulfo-NHS to give the reactive NHS ester.

1. Light-emitting nanoparticle nuclei with reactive amine groups as described in Example 1 were reacted with succinic anhydride to form a carboxyl group at the particle core surface and 2.5 mg of the nanoparticles as a suspension in methanol was aliquoted into a microcentrifuge tube. The suspension was centrifuged at 14,000 rpm for 4 min; the solvent decanted and the pellet resuspended in 500 μL of MES buffer with ultrasonication.

2. 60 μL of a 200 mM N-hydroxysulfosuccinimide [sulfo-NHS] in MES buffer was added to the nanoparticles.

3. 6 μL of a 200 mM N-(3-dimethylaminopropyl)-N′-ethylcarbodiimide hydrochloride [EDC] in MES buffer was added to the nanoparticles, and the suspension agitated at room temperature for 30 min.

4. The nanoparticles were pelleted by centrifugation (14,000 rpm, 4 min), the supernatant was decanted and the pellet resuspended in 500 μL MES with ultrasonication.

5. Step 4 was repeated a further 3 times; on the final step the pellet was resuspended in 200 μL MES with ultrasonication.

6. While the nanoparticles were agitating, 300 μL of 0.5 mg/mL goat anti-mouse antibody (clone Poly4053, purchased from Biolegend) was desalted into MES using a zeba column.

7. Immediately after completing the nanoparticles washings (step 5), the desalted antibody was added to the nanoparticles and the suspension agitated for 2 h.

8. 124 μL of a 1.0 M glycine solution in MES was added to the nanoparticles to quench any remaining NHS-activated functionality on the nanoparticles surface. The suspension was agitated for 30 min.

9. The nanoparticles were pelleted by centrifugation (14,000 rpm, 4 min), the supernatant was decanted and the pellet resuspended in 500 μL BSA/MES with ultrasonication.

10. The suspension was agitated at room temperature for 1 h, before being transferred to the fridge overnight, after which step 9 was repeated a further 2 times.

### Comparative Example 2—Click Chemistry Conjugation of Particles

Bioconjugation using a metal-free click reaction between the strained alkyne dibenzocyclooctyne [DBCO] and an azide group was used as described in Beilstein J. Org. Chem. 2018, 14, 11-24 and Polym. Chem. 2019, 10, 705-717).

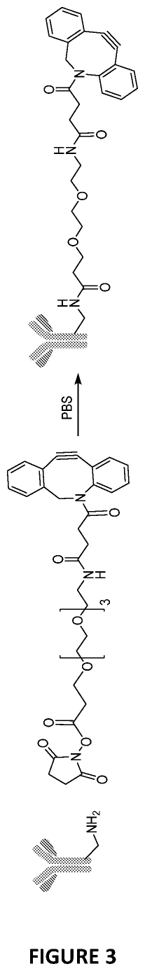

DBCO-functionalised goat anti-mouse IgG was prepared using a small molecule heterobifunctional linker featuring DBCO and NHS ester end groups as illustrated in FIG. 3.

The NHS ester of the DBCO linker reacts with protein lysine chains in PBS, and the resulting antibody conjugate is purified from excess linker using spin filtration.

The number of DBCO linkers per IgG can be varied by changing the number of molar equivalents of the DBCO linker to IgG. The number of DBCO linkers in the purified conjugate is measured by UV-vis: DBCO has a λₘₐₓ at 309 nm (c=12,000 M⁻¹ cm⁻¹) and IgG has a λₘₐₓ at 280 nm (c=204,000 M⁻¹ cm⁻¹). The activity of the DBCO-labelled antibody conjugate relative to native unconjugated antibody was assayed as a function of number of DBCO linkers per antibody. In this way, the number of linkers per IgG was varied between 6-24, with the optimum <10 linkers/IgG.

The DBCO linkers were used to link the antibody to a light-emitting marker nanoparticle according to the following procedure:
1. 6.5 μL of a freshly prepared 10 mM solution of dibenzylcyclooctyne-PEG4-NHS ester (DBCO-PEG4-NHS purchased from Jena Bioscience, product code CLK-A134) in anhydrous DMSO was added to 195 μL of goat anti-mouse IgG (1 mg/mL solution as supplied by the manufacturer) and the mixture was agitated for 30 min at room temperature.
2. 10.1 μL of 1 M Tris hydrochloride buffer, pH 8 was added to quench any unreacted DBCO-PEG4-NHS linker, and the solution agitated for 5 minutes.
3. The mixture was desalted into PBS using a zeba column.
4. The number of DBCO linkers per antibody was quantified using UV-vis and stored at 4° C. until conjugated with azide-functionalised nanoparticles (steps 5-8).
5. Nanoparticles were formed as described in Example 1, except that azide-PEG-COOH, illustrated below, (Mw 2,000 g/mol) was used in place of biotin-PEG-COOH. 1 mg of 1% azide-functionalised 80 nm nanoparticles as a suspension in methanol was aliquoted into a microcentrifuge tube. The suspension was centrifuged at 14,000 rpm for 4 min; the solvent decanted and the pellet resuspended in 1 mL of BSA/PBS with ultrasonication.
6. 136 μL of the DBCO functionalised antibody described above was added, and the mixture agitated at room temperature for 4 h.
7. The nanoparticles were pelleted by centrifugation (14,000 rpm, 4 min), the supernatant was decanted and the pellet resuspended in 1 mL BSA/PBS with ultrasonication.
8. Step 3 was repeated a further 3 times; on the final step the pellet was resuspended in 100 μL BSA/PBS with ultrasonication.

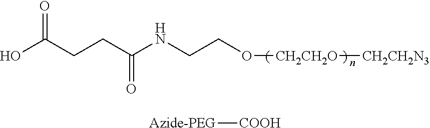

Plate Assays

Assay plates were imaged on an Olympus BX60 upright microscope in a dark room facility, using the UV channel and a 10× magnification lens. Integration time (in ms) was set to maximise the emission of a 2 mg/mL (+) well without saturation of the detector, and this was used for all subsequent measurements of a given plate. A background measurement was also taken at the measurement length and subtracted from each measured well emission. Light-emission intensity arising from the light-emitting polymer in the nanoparticles for each well is the integral of emission between 400-600 nm. Signal-to-noise is calculated as the background corrected average of the (+) well emission intensity for a given concentration, divided by the background corrected average of the (—) well emission intensity for the same concentration.

### Assay Using Example 2 Nanoparticles

The nanoparticles of Example 2 were assayed against (+) mouse anti-human CD4 and (−) BSA at concentrations of 0.5, 1 and 2 mg/ml of the nanoparticles.

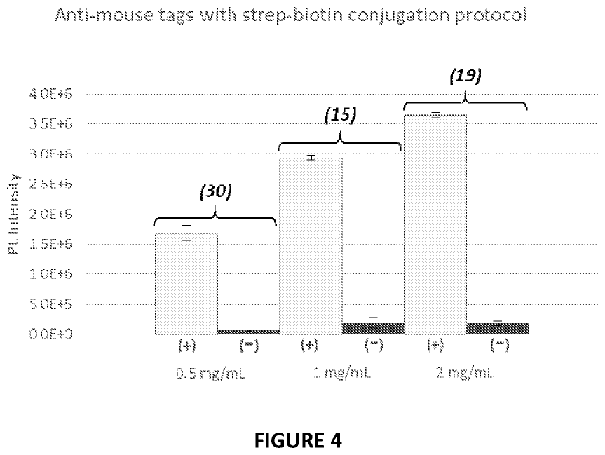

With reference to FIG. 4, the assay results show an increasing positive signal with nanoparticle concentration, with minimal background on the negative wells, resulting in a large signal to noise ratio (shown in parentheses). The fluorescence standard deviation for each concentration is low.

Assay using Comparative Example 1 nanoparticles:

For the EDC/NHS conjugated nanoparticles of Comparative Example 1 various conjugation reaction parameters were screened to maximise assay signal-to-noise:
Buffer pH and ionic strength;
Antibody concentration in the reaction;
Use of Trizma (2-amino-2-(hydroxymethyl)-1,3-propanediol) in place of glycine to quench excess surface NHS-ester following IgG reaction

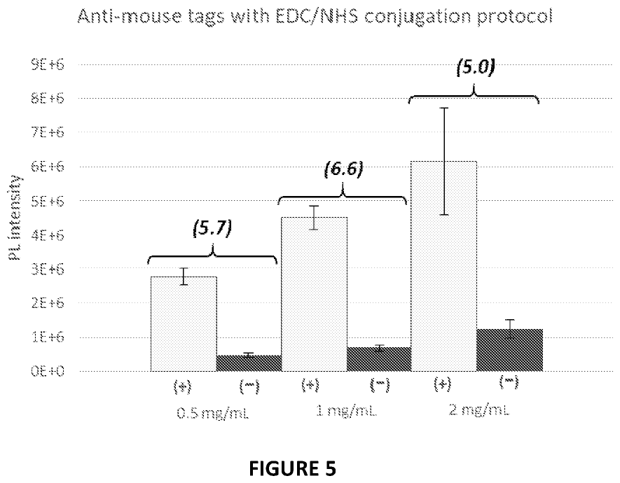

The assay results for the best of these conditions are shown in FIG. 5.

The maximum positive signal for the EDC/NHS conjugation particles of Comparative Example 1 (6.2×10⁶) is comparable to that of the streptavidin-biotin particles of Example 1 at the same concentration (3.6×10⁶). However, the background in the negative wells for the particles of Comparative Example 1 is an order of magnitude higher than that of the streptavidin-biotin exemplary particles (1.2×10⁶ vs. 1.9×10⁵).

In addition, an increased standard deviation of the fluorescence intensity is seen, suggesting an uneven distribution of particles across the well surface, indicating that the particles are aggregating under the assay conditions.

### Assay Using Comparative Example 2 Nanoparticles

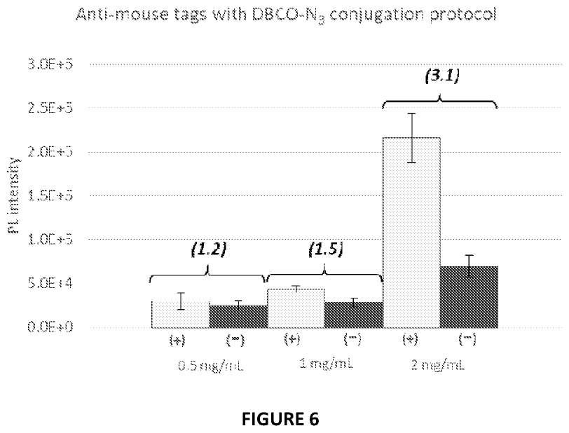

The assay results for DBCO-N3 conjugated IgG-nanoparticles of Comparative Example 2 are shown in FIG. 6. The results differ considerably from that of the streptavidin-biotin conjugation particles of Example 1: at 0.5 mg/mL and 1 mg/mL concentrations, the difference between the positive and negative well signals is negligible, and the resulting signal-to-noise ratio is <2. While a large jump in the positive signal is seen at 2 mg/mL, the photoluminescent intensity is an order of magnitude lower than that of the streptavidin-biotin conjugated particles of Example 1 at the same concentration (2.2×10⁵ for DBCO-N3 vs. 3.6×10⁶ for streptavidin-biotin). A rise in photoluminescent intensity is also seen for the negative wells at 2 mg/mL; as a result the signal-to-noise ratio at this concentration is also poor at 3.1.

Flow Cytometry Assays

Flow cytometry analysis was carried out on a Propel Labs YETI analyser.

BV421-anti-human CD4 (clone SK3) [BV421-hCD4] and BV421-mouse IgG1, κ isotype (clone MOPC-21) [BV421-isotype] were purchased from Biolegend.

CYTO-TROL cells were supplied by Beckman Coulter.
1. CYTO-TROL cells were brought up to room temperature and resuspended in the buffer supplied by the manufacturer before use.
2. Antibody-tag dilutions were made to the required concentrations in BSA/PBS in microcentrifuge tubes.
3. 100 μL of prepared Cyto-Trols were added to each of the antibody-NP dilutions. The preparations were mixed well and left to incubate at 4° C. for 30 minutes, avoiding direct light.
4. After incubation, the cells were washed twice with cell staining buffer, centrifuged at 2000 rpm for 3 minutes at 4° C., and the supernatant was discarded.
5. The cells were resuspended in 200 μL cell staining buffer ready for analysis.
6. For analysis, a flow rate of 0.5 μL/sec was used. Data was gated on single cells, and 10,000 events were acquired in this gate for each measurement.

A series of different exemplary nanoparticles NP(x)-hCD4 (where NP is a nanoparticle as described in Example 1, x=s for streptavidin; x=n for neutravidin; hCD4=anti-human CD4 antibody) dilutions were screened to determine the optimal working concentration for maximum staining index of each dye.

Conjugation of antibodies using neutravidin is as described above for streptavidin except that 50 μL of a 1 mg/mL solution of neutravidin in PBS was used in place of streptavidin.

Staining index [SI] is:

SI=(MFI1−MFI2)/(2×SD)

where MFI1 is the median fluorescence intensity of the positive population; MFI2 is the median fluorescence intensity of the negative population, and SD is the standard deviation of the negative signal.

The same was done for comparative marker BV421-hCD4 wherein BV421 is the dissolved fluorescent polymer Brilliant Violet 421™ available from BioLegend.

Voltages were adjusted to ensure both negative and positive signals were on scale. For NP(n)-hCD4 it was not possible to find a voltage where negatives and positives were fully on scale, so voltages where the negatives were fully on scale were used for analysis.

TABLE 1

Tag

| | BV421-hCD4 | NP(x)-hCD4 |
|---|---|---|
| Concentrations | 500 | 125 |
| screened (μg/mL) | 250 | 62.5 |
| | 125 | 31.3 |
| | 62 | 15.6 |
| | 31 | 7.8 |

Optimal concentrations for each tag were determined as:
BV421-hCD4: 250 μg/mL
NP(s)-hCD4: 31.3 μg/mL
NP(n)-hCD4: 15.6 μg/mL

The optimal voltages for BV421-hCD4, NP(s)-hCD4 and NP(n)-hCD4 for each channel were the same, and are summarised in Table 2:

TABLE 2

| Emission Channel | |
|---|---|
| (Laser line nm) | Voltage/V |
| FSC | 370 |
| SSC | 478 |
| 420/10 (405) | 470 |
| 460/22 (405) | 421 |
| 525/50 (405) | 380 |
| 615/24 (405) | 471 |
| 525/35 (488) | 460 |
| 593/52 (488) | 424 |
| 692/80 (488) | 520 |
| 750LP (488) | 630 |

Each of the following tags of Table 3 were measured in triplicate at their optimum concentration and optimised voltages as described above:

TABLE 3

| Tag type | Tag | Controls |
|---|---|---|
| BV421 | BV421-hCD4 | BV421-isotype |
| NP(s) | NP(s)-hCD4 | NP(s)-isotype |
| NP(n) | NP(n)-hCD4 | NP(n)-isotype |

The same was done for the biotinylated nanoparticle of Example 1 (i.e. without streptavidin or neutravidin) and unstained cells.

These particles not conjugated to streptavidin or neutravidin were aliquoted into a microcentrifuge tube. The suspension was centrifuged at 14,000 rpm for 4 min; the solvent decanted and the pellet resuspended in 100 μL of BSA/PBS with ultrasonication to give a final particle concentration of 10 mg/mL.

Results: NP(s)-hCD4

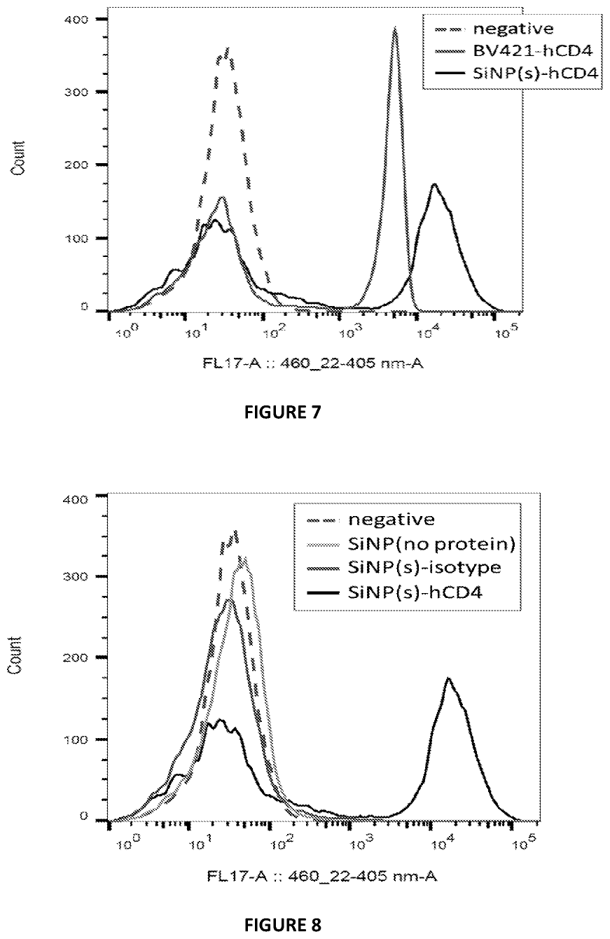

The staining of Cyto-Trol cells with NP(s)-hCD4 is compared with BV421-hCD4 in FIG. 7 and Table 4. Similar to BV421-hCD4, NP(s)-hCD4 shows negligible staining of negative cells. The positive signal for NP(s)-hCD4 however is significantly shifted to higher extinction coefficients, with only a slight increase in the standard deviation of the positive signal relative to BV421-hCD4. This increase in MFI1 leads to a ˜2.5× increase in staining index for NP(s)-hCD4 relative to BV421.

TABLE 4

| | Brightness | Background | |
|---|---|---|---|
| Tag | (MFI1-MFI2) | (2 × SD) | Staining Index |
| NP(s)-hCD4 | 17874 | 57 | 314 |
| BV421-hCD4 | 4834 | 38 | 127 |

The low negative staining of NP(s)-derived particles are further demonstrated in FIG. 8 and Table 5. Neither the unfunctionalised, NP(s)-isotype or the NP(s)-hCD4 show any significant negative staining, showing that fluorescent nanoparticles and their streptavidin- and antibody-conjugated derivatives as described herein have low non-specific absorption to cells.

TABLE 5

| | Background |
|---|---|
| Experiment | (2 × SD) |
| Negative | 35 |
| NP(no protein) | 57 |
| NP(s)-isotype | 48 |
| NP(s)-hCD4 | 57 |

Results: Np(n)-Hcd4

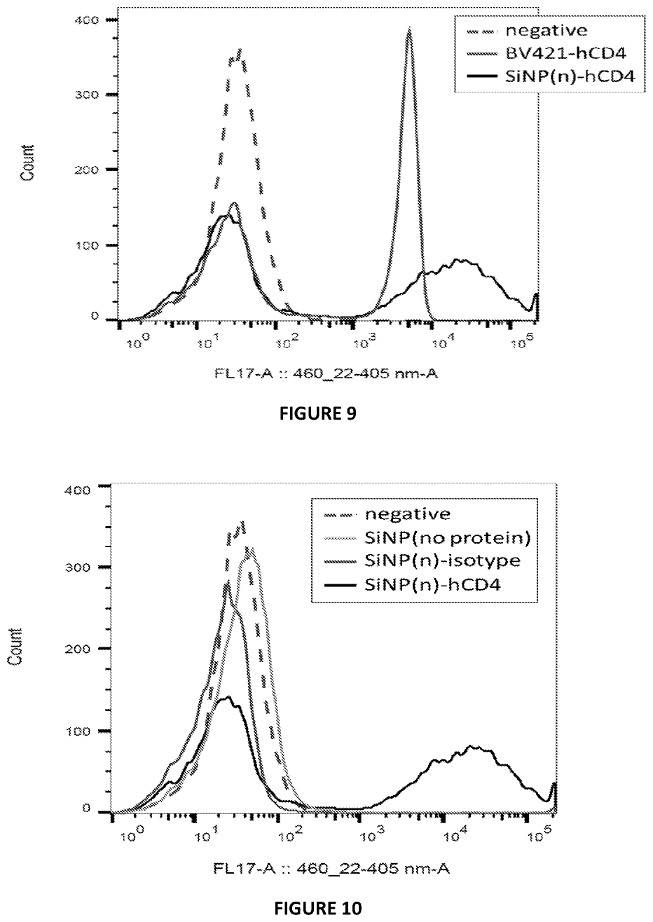

As for NP(s)-hCD4, the staining of Cyto-Trol cells with NP(n)-hCD4 is compared with BV421-hCD4 in FIG. 9 and Table 6. As with NP(s)-hCD4, NP(n)-hCD4 shows negligible negative staining. The brighter positive signal for NP(n)-hCD4 gives a staining index of 5.4× relative to BV421-hCD4.

TABLE 6

| | Brightness | Background | |
|---|---|---|---|
| Tag | (MFI1-MFI2) | (2 × SD) | Staining Index |
| NP(n)-hCD4 | 29376 | 43 | 681 |
| BV421-hCD4 | 4834 | 38 | 127 |

The low negative staining of NP(n)-derived particles are further demonstrated in FIG. 10 and Table 7. Neither the unfunctionalised, NP(n)-isotype or the NP(n)-hCD4 show any significant negative staining, showing that fluorescent NPs and their neutravidin- and antibody-conjugated derivatives have low non-specific absorption to cells.

TABLE 7

| | Background |
|---|---|
| Experiment | (2 × SD) |
| Negative | 35 |
| NP(no protein) | 57 |
| NP(n)-isotype | 32 |
| NP(n)-hCD4 | 43 |

Effect of First Biotin Group Density

The effect of the first biotin group density (and therefore the number of protein binding sites on the surface of the precursor nanoparticle) was studied using nanoparticles as described in Examples 1 and 2 except that biotin-PEG-COOH as a percentage of the total weight of biotin-PEG-COOH+SAA-PEG-SAA was varied to 0.5 wt %, 1 wt %, 5 wt % and 10 wt %.

Volumes of reagents used for different weight percentages of the first and second surface groups are shown in Table 8:

TABLE 8

| | (A) | | | |
|---|---|---|---|---|
| | Nanoparticle | | (C) | (D) |
| | volume for | (B) | Nanoparticle | Biotinylated |
| Wt % | streptavidin | Volume of | volume for | antibody |
| Biotin | conj./μL | streptavidin/μL | IgG conj./μL | volume/μL |
| 0.5 | 731 | 18.8 | 656 | 93.7 |
| 1 | 713 | 37.5 | 563 | 188 |
| 5 | 563 | 188 | 506 | 244 |
| 10 | 375 | 375 | 255 | 495 |

Nanoparticles conjugated to anti-human CD4 [NP(x)-hCD4] were formed using biotin mouse anti-human CD4 antibody (clone SK3) supplied by Biolegend.

Isotype control particles [NP(x)-isotype] used biotin mouse IgG1, κ isotype (clone MOPC-21) supplied by Biolegend.

A series of different NP(s)-x % hCD4 (where x=0.5, 1, 5 or 10, representing the percentage PEG-biotin used to functionalise the NP) and BV421-hCD4 dilutions were screened to determine the optimal working concentration for maximum staining index of each dye.

Voltages were adjusted to ensure both negative and positive signals were on scale.

TABLE 9

Tag

| | BV421-hCD4 | NP(s)-x % hCD4 |
|---|---|---|
| Concentrations | 500 | 500 |
| screened (μg/mL) | 250 | 250 |
| | 125 | 125 |
| | 62 | 62 |
| | 31 | 31 |

Optimal concentrations for each tag were determined as:
BV421-hCD4: 250 μg/mL
NP(s)-0.5% hCD4: 62.5 μg/mL
NP(s)-1% hCD4: 250 μg/mL
NP(s)-5% hCD4: 125 μg/mL
NP(s)-10% hCD4: 62.5 μg/mL

The optimal voltages for BV421-hCD4 and NP(s)-x % hCD4 for each channel were the same, and are summarised in Table 10:

TABLE 10

| Emission Channel | |
|---|---|
| (Laser line) | Voltage/V |
| FSC | 370 |
| SSC | 478 |
| 420/10 (405) | 470 |
| 460/22 (405) | 421 |
| 525/50 (405) | 380 |
| 615/24 (405) | 471 |
| 525/35 (488) | 460 |
| 593/52 (488) | 424 |
| 692/80 (488) | 520 |
| 750LP (488) | 630 |

Tags of Table 11 were measured in triplicate at their optimum concentration and optimised voltages as described above:

TABLE 11

| Tag type | Tag | Controls |
|---|---|---|
| BV421 | BV421-hCD4 | BV421-isotype |
| NP(s)-0.5% | NP(s)-0.5% hCD4 | NP(s)-0.5% isotype |
| NP(s)-1% | NP(s)-1% hCD4 | NP(s)-1% isotype |
| NP(s)-5% | NP(s)-5% hCD4 | NP(s)-5% isotype |
| NP(s)-10% | NP(s)-10% hCD4 | NP(s)-10% isotype |

Unstained cells were also measured.

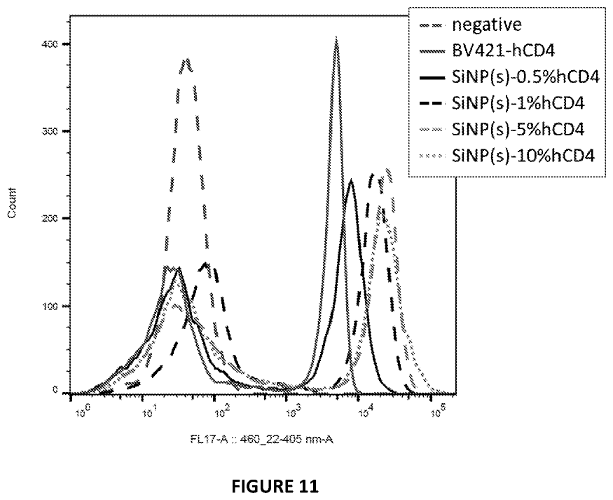

The effect of changing the percentage of surface biotin groups on the staining of Cyto-Trol cells is illustrated in FIG. 11 and Table 12. For all biotin concentrations, the negative population showed negligible background staining relative to unstained cells, with the exception of 1% biotin, which is attributed to the very high optimal staining concentration (250 μg/mL) compared to the other tags. The effect of this high staining concentration is apparent from the NP(s)-1% hCD4 used in the streptavidin/neutravidin comparison experiments above which show negligible staining for the negative population at the much lower optimal staining concentration of 31.3 μg/mL.

Therefore, it can be concluded that variation of the surface biotin concentration between 0.5-10% does not increase the non-specific binding of the resulting tag to Cyto-Trol cells.

TABLE 12

| | Brightness | Background | |
|---|---|---|---|
| Tag | (MFI1-MFI2) | (2 × SD) | Staining Index |
| BV421-hCD4 | 4731 | 50 | 107 |
| NP(s)-0.5% hCD4 | 7254 | 57 | 127 |
| NP(s)-1% hCD4 | 16043 | 110 | 146 |
| NP(s)-5% hCD4 | 21908 | 96 | 229 |
| NP(s)-10% hCD4 | 21517 | 90 | 240 |

These data demonstrate that more surface biotin results in a higher staining index of the resulting antibody conjugate, with 10 wt % biotin tags showing a 1.9×higher staining index than 0.5 wt % biotin tags. Even at 0.5 wt % biotin, the staining index exceeds that of BV421-hCD4.

Comparative Indirect Staining Flow Cytometry Experiment Using Streptavidin-Modified Nanoparticles

Precursor light-emitting nanoparticles functionalised with streptavidin were formed as described in Examples 1 and 2, i.e. without conjugation of the streptavidin to a biotinylated antibody. Following conjugation to streptavidin, excess streptavidin was removed by centrifuging at 14,000 rpm for 3.5 min and carefully removing the supernatant with a pipette. For storage, the pelleted NP-streptavidin precursor nanoparticles were resuspended by sonication in phosphate buffered saline (pH 7.4) containing 1 wt. % BSA to give a final nanoparticle concentration of 10 mg/mL.

The optimal staining concentration for biotin mouse anti-human CD4 (clone SK3) purchased from Biolegend was determined by incubating 100 μL of CYTO-TROL cells supplied by Beckman Coulter at 4° C. for 30 min with the concentrations of antibody shown in Table 13. After this, the cells were washed twice with cell staining buffer, centrifuged at 2,000 rpm for 3 minutes at 4° C., and the supernatant was discarded. After resuspending in 100 μL cell staining buffer, cells were incubated 4° C. for 30 min with 500 μg/mL of NP-streptavidin and washed as above. After this, the cells were resuspended in 200 μL of cell staining for analysis. For analysis, a flow rate of 0.5 μL/sec was used. Data was gated on single cells, and 10,000 events were acquired in this gate for each measurement.

0.075 μg/mL of biotinylated antibody was selected as the optimal antibody concentration, since this gave the lowest background staining to non-CD4 cells in the sample as well as a comparatively high staining index (see table 13).

TABLE 13

| Primary | | Background | Staining |
|---|---|---|---|
| antibody conc | Brightness | 2 × standard | index |
| (μg/mL) | [MFI1-MFI2] | deviation | (SI) |
| 0.5 | 27427 | 145 | 190 |
| 0.25 | 13453 | 89 | 150 |
| 0.125 | 10486 | 65 | 162 |
| 0.075 | 14382 | 78 | 183 |

The procedure above was repeated, but with a fixed biotinylated antibody concentration of 0.075 μg/mL and varying NP-streptavidin concentrations (as shown in table 14). Side-by side comparative experiments were also carried out using the dissolved fluorescent polymer BV421 streptavidin at the concentrations shown in table 14. A PMT voltage of 424 V was used when acquiring data for all flow cytometry experiments. Data was gated on single cells, and 10,000 events were acquired in this gate for each measurement.

The data set out in Table 14 shows that although NP-streptavidin particles have a higher value of [MFI1-MFI2] at all measured concentrations as compared to BV421, their significantly higher background results limits their staining index.

TABLE 14

| | | | | Staining |
|---|---|---|---|---|
| | Concentration | | 2 × standard | index |
| Tag | (μg/mL) | [MFI1-MFI2] | deviation | (SI) |
| NP- | 1500 | 10689 | 108 | 99 |
| streptavidin | | | | |
| NP- | 1250 | 10208 | 107 | 95 |
| streptavidin | | | | |
| NP- | 1000 | 10715 | 86 | 125 |
| streptavidin | | | | |
| NP- | 750 | 12520 | 79 | 158 |
| streptavidin | | | | |
| NP- | 500 | 13112 | 65 | 202 |
| streptavidin | | | | |
| BV 421 | 1 | 6113 | 21 | 297 |
| BV 421 | 0.75 | 6488 | 23 | 277 |
| BV 421 | 0.5 | 5790 | 20 | 287 |
| BV 421 | 0.25 | 4665 | 20 | 229 |
| BV 421 | 0.1 | 3028 | 17 | 179 |

1. An assay method for a target analyte comprising contacting a sample with a light-emitting marker and determining any binding of the target analyte to the light-emitting marker wherein the light-emitting marker comprises a light-emitting core comprising a light-emitting material; a first group bound to the light-emitting core and comprising a first biotin group; a second biotin group bound to a biomolecule, and a protein bound to the first and second biotin groups.

2. The assay method according to claim 1 wherein the biomolecule comprises an antigen-binding fragment.

3. The assay method according to claim 2 wherein the biomolecule is an antibody.

4. The assay method according to claim 1 wherein the protein is selected from avidin, streptavidin, neutravidin and recombinant variants thereof.

5. The assay method according to claim 1 wherein the light-emitting material is a light-emitting polymer.

6. The assay method according to claim 1 wherein the light-emitting marker is dispersed in the sample.

7. The assay method according to claim 1 wherein the light-emitting marker is a light-emitting particle comprising a particulate light-emitting core comprising the light-emitting material and wherein a first surface group bound to a surface of the light-emitting particle core comprises the first biotin group.

8. The assay method according to claim 7 wherein the first surface group comprises a polyether chain disposed between the surface of the light-emitting particle core and the first biotin group.

9. The assay method according to claim 8 wherein the polyether group is a group of formula (I):
—((CR¹⁴R¹⁵)_{b}O)_{c}— (I)
wherein R¹⁴ and R¹⁵ are each independently H or C₁₋₆ alkyl; b is at least 1; and c is at least 2.

10. The assay method according to claim 7 comprising a second surface group bound to the surface of the light-emitting core wherein the second surface group does not comprise biotin.

11. The assay method according to claim 10 wherein the second surface group comprises a polyether.

12. The assay method according to claim 10 wherein the first surface group: second surface group molar ratio is in the range of 1:1000-1:10.

13. The assay method according to claim 7 wherein the light-emitting particle core comprises the light-emitting material and a matrix material.

14. The assay method according to claim 13 wherein the matrix material is silica.

15. The assay method according to claim 1 wherein the light-emitting marker is dissolved in the sample.

16. (canceled)

17. The assay method according to claim 1 any one of the preceding claims wherein the sample contacted with the light-emitting marker is analysed by flow cytometry.

18. (canceled)

19. The assay method according to claim 1 wherein the sample comprises a mixture of cells and one or more different types of target cells bound to the light-emitting marker are identified and/or quantified.

20. A light-emitting marker comprising a light-emitting core comprising a light-emitting material; a first group bound to the light-emitting core and comprising a first biotin group; a second biotin group bound to a biomolecule, and a protein bound to the first and second biotin groups.

21. A colloid comprising light-emitting marker particles according to claim 7 suspended in a liquid.

22-23. (canceled)

24. A method of forming a light-emitting marker according to claim 20, the method comprising contacting the biomolecule bound to the second biotin group with a precursor light-emitting marker comprising the first biotin group and the protein bound to the first biotin group.

25. (canceled)
